(19) United States
(12) Patent Application Publication
(10) Pub. No.: US 2007/0233251 A1
(43) Pub. Date: Oct. 4, 2007

(54) Use of Magnetic Fields in Orthopedic Implants

(76) Inventor: M. S. Abdou, San Diego, CA (US)

Correspondence Address: FISH & RICHARDSON, PC, P.O. BOX 1022, MINNEAPOLIS, MN, 55440-1022, US

(21) Appl. No.: 11/677,008

(22) Filed: Feb. 20, 2007

Related U.S. Application Data
(60) Provisional application No. 60/774,519, filed on Feb. 18, 2006.

(51) Int. Cl. A61F 2/44

(52) U.S. Cl. 623/17.11

(57) ABSTRACT

An orthopedic device is adapted to be implanted between a first bone and a second bone of a skeletal structure. The device includes magnetically charged members that emit magnetic fields that determine the interaction of members of the device.

### REFERENCE TO PRIORITY DOCUMENT

This application claims priority of co-pending U.S. Provisional Patent Application Ser. No. 60/774,519 filed Feb. 18, 2006. Priority of the aforementioned filing date is hereby claimed and the disclosure of the Provisional Patent Application is hereby incorporated by reference in its entirety.

### BACKGROUND

Pain from degenerative joint disease is a major health problem in the industrialized world and replacement of the degenerating joint is emerging as the preferred treatment strategy in these patients. Removal of the painful joint and replacement with a mobile prosthesis is an intuitive and highly successful treatment option. Because of the aging population, these operations are being performed in an increasing number of patients. Despite the success of joint replacement surgery, implant failure remains a significant problem. Wear of the implant components and device loosening from the underlying bone have emerged as the most common reasons for device failure. Implant replacement with a second operation is more technically difficult, more costly, has a higher complication rate and a lower probability of success than the initial joint replacement procedure. Thus, it is highly advantageous that implant longevity be maximized.

Overall, the encouraging experience with the mobile hip prosthesis has lead to development of prosthetic joints for use in the knee, shoulder, ankle, digits and other joints of the extremities. The vast experience with these devices has again shown that the wear debris produced by the bearing surfaces and the loosening that occur at the bone-device interface are major causes of implant failure. The latter is at least partially caused by the former, since it's been shown that the particulate debris from the bearing surfaces promote bone re-absorption at the bone-device interface and significantly accelerates device loosening. In the long term, the degradation products of the implant materials may also produce negative biological effects at distant tissues within the implant recipient.

While ceramic and polymer implant components produce wear debris, these degradation products are usually deposited as insoluble particles around the implant thereby limiting the extent of potential toxicity. In contrast, metallic degradation products may be present as particulate and corrosion debris as well as free metals ions, composite complexes, inorganic metal salts/oxides, colloidal organo-metallic complexes and other molecules that may be transported to distant body sites. In fact, studies have revealed chronic elevations in serum and urine cobalt and chromium level after prosthetic joint replacement. Given the known toxicity of titanium, cobalt, chromium, nickel, vanadium, molybdenum and other metals used in the manufacture of orthopedic implants, the tissue distribution and biologic activity of their degradation products is of considerable concern. Host toxicity may be produced directly by the reactive metallic moieties as well as by their alterations of the immune system, metabolic function, and their potential ability to cause cancer. These issues are thoroughly discussed in the text “Implant Wear in Total Joint replacement” edited by Thomas Wright and Stuart Goodman and published by the American Academy of Orthopedic Surgeons in 2000. The text is hereby incorporated by reference in its entirety.

More recently, joint replacement has been attempted in the spine. Because each of the twenty three motion segments between the second cervical vertebra and the sacrum contains three joints, there is a vast potential for the use of joint replacement technology in the spine. Unlike joints in the extremities, proper function of the spinal joints (e.g., inter-vertebral disc and facet joints) returns the attached bones to the neutral position after the force producing the motion has dissipated. That is, a force applied to the hip, knee or other joints of the extremities produces movement in the joint and a change in the position of the attached bones. After the force has dissipated, the bones remain in the new position until a second force is applied to them. In contrast, the visco-elastic properties of the spinal disc and facet joint capsule dampen the force of movement and return the vertebral bones to a neutral position after the force acting upon them has dissipated.

Prosthetic joint implants that attempt to imitate native spinal motion have usually employed springs, memory shape materials, polyurethane, rubber and the like to recreate the visco-elastic properties of the spinal joints. U.S. Pat. Nos. 4,759,769; 5,674,296; 5,976,186; 6,022,376; 6,093,205; 6,348,071; 6,761,719; 6,966,910 (all of which are herein incorporated by reference in their entirety) and others disclose some of these spinal implants. When subjected to the millions of cycles of repetitive loading that is required of a spinal joint prosthesis, all implants to date have been plagued by excessive wear and degeneration secondary to the fairly modest wear characteristics of these elastic elements. Thus, in addition to the wear debris generated by the bearing surface(s), the elastic materials used to recreate spinal motion will produce a second source of degradation products. Given the number of joints in the spine and the extensive potential application of replacement technology in these joints, it is critical that the wear debris from the implanted prosthesis be minimized.

### SUMMARY

The preceding discussion illustrates a continued need in the art for the development of mobile orthopedic prosthesis' with a reduced wear profile. This development would maximize the functional life of the prosthesis and minimize the production of degradation products and their potential toxicity.

Various orthopedic implants are disclosed herein. The wear characteristics of the implant are at least partially determined by the material of composition, the coefficient of friction and the load borne by the bearing surface. The first two variables have been extensively studied and manipulated. In the disclosed devices, magnetic fields are used to alter the bearing surface load within the device. One or more elements of the mobile prosthesis produce a magnetic field and the prosthesis is constructed in such a way so as to produce attraction/repulsion forces between the prosthesis sub-segments. The magnetic fields are used to partially or completely separate and unload the articulating surfaces of the prosthetic joint. This feature minimizes the contact between the articulating surfaces, thereby increasing device longevity and producing a lesser quantity of toxic wear debris.

In another application, a neutral configuration of the orthopedic implant exists in which the various forces acting upon the mobile prosthesis are in relative balance. Movement of the prosthesis away from the neutral position produces an imbalance in the sum of forces and causes the prosthesis to oppose any movement away from that neutral position. After the force acting upon the prosthesis has dissipated, the implant returns the attached bones to the neutral position. Unlike prior art, use of magnetic fields can recreate the visco-elastic motion characteristics of the native spine without the use of elastomers or mechanical means that produce degradation products.

In another application, magnetic fields are used to increase the holding power of an internal locking mechanism within an orthopedic implant. In another application, the magnetic fields themselves are used to treat the painful surrounding tissues. U.S. Pat. No. 6,524,233; 6,447,440; 6,119,631; 6,048,302; 5,842,966; 5,669,868; 5,665,049; 5,453,073; 5,387,176; 5,131,904; and other illustrate the therapeutic use of magnetic fields. The fields generated by the magnetic members of the implant may be used to reduce the pain within the neighboring tissues. Since variable magnetic fields have been shown to provide a greater therapeutic effect on surrounding tissues than magnetic fields of constant value, the static fields produced by the fixed implant magnets may be varied. While this can be done by using electro-magnets with pulsatile variation in field strength, it can also be done using a mobile magnetic shield on a fixed magnet. For example, a member of the prosthesis that is mobile relative to the magnetic field source can be fitted with magnetically shielding material and positioned between the field source and the target tissue. With normal prosthesis movement, the shielding member will move between the magnetic member and the surrounding tissues and the tissues will experience a variation in the magnetic field.

In one aspect, there is disclosed an orthopedic device adapted to be implanted between a first bone and a second bone of a skeletal structure, comprising: a first member having an abutment surface adapted to contact a surface of the first bone, wherein the first member emits a first magnetic field of a first polarity; a second member having an abutment surface adapted to contact a surface of the second bone, wherein the second member emits a second magnetic field of the same polarity as the first polarity; and at least one bearing member between the first and second members that permits relative movement between the first and second members and that bears a load between the first and second members, wherein the load on the bearing surface is reduced as a result of an interaction of the magnetic fields.

In another aspect, there is disclosed an orthopedic device adapted to be implanted between a first bone and a second bone of a skeletal structure, comprising: a first abutment member having an abutment surface adapted to contact a surface of the first bone; a first magnetic member at least partially contained within the first abutment member, wherein the first magnetic member emits a first magnetic field of a first polarity; a second abutment member having an abutment surface adapted to contact a surface of the second bone; and a second magnetic member at least partially contained within the second abutment member, wherein the second magnetic member emits a second magnetic field of the same polarity as the first polarity; wherein the first and second abutment members have a spatial relationship that is at least partially determined by an interaction of the first and second magnetic fields.

In another aspect, there is disclosed an orthopedic device adapted to be implanted between a first bone and a second bone of a skeletal structure, comprising: a first abutment member having an abutment surface adapted to contact a surface of the first bone; a first magnetic member at least partially contained within the first abutment member, wherein the first magnetic member emits a first magnetic field; a second abutment member having an abutment surface adapted to contact a surface of the second bone; and a second magnetic member at least partially contained within the second abutment member, wherein the second magnetic member emits a second magnetic field; wherein the first and second abutment members have a default spatial relationship and wherein movement of the first and second members away from the default spatial relationship is opposed by interaction of the first and second magnetic fields.

In another aspect, there is disclosed an orthopedic device adapted to be implanted between a first bone and a second bone of a skeletal structure, comprising: a first abutment member having an abutment surface adapted to contact a surface of the first bone; a first magnetic member at least partially contained within the first abutment member, wherein the first magnetic member emits a first magnetic field; and a second abutment member having an abutment surface adapted to contact a surface of the second bone; a second magnetic member at least partially contained within the second abutment member, wherein the second magnetic member emits a second magnetic field; wherein the first and second members can move relative to one another and wherein relative movement between the first and second members is at least partially hindered by interaction of the magnetic fields.

In another aspect, there is disclosed an orthopedic device adapted to be implanted in a patient, comprising: a first member having an abutment surface adapted to attach to a surface of a bone so as to aid in segmental stabilization of the patient's skeletal system; and a first magnetic member at least partially contained within the first abutment member, wherein the first magnetic member emits a first magnetic field such that the magnetic field reaches a tissue of the patient.

Other features and advantages will be apparent from the following description of various embodiments, which illustrate, by way of example, the principles of the disclosed devices and methods.

### BRIEF DESCRIPTION OF THE DRAWINGS

FIG. 1 shows a perspective view of a first embodiment of an implant that is sized and shaped to be positioned within a disc space between a pair of vertebrae in a spine.

FIGS. 2A and 2B show exploded views of the implant of FIG. 1.

FIG. 3 shows a cross-sectional view of the implant of FIG. 1.

FIG. 4 shows another embodiment of an implant that includes upper and lower components.

FIGS. 5 and 6 schematically show arrangements of magnets within orthopedic implants.

FIG. 7 shows another embodiment of an implant.

FIGS. 8A and 8B shows exploded views of the implant of FIG. 7.

FIGS. 9 and 10 shows cross-sectional views of the implant of FIG. 7.

FIG. 11 shows a dynamic screw assembly in an assembled state.

FIG. 12A shows the dynamic screw assembly in an exploded state.

FIGS. 12B and 12C show dynamic screw assemblies attached to vertebral bodies V1 and V2 and linked via a rod.

FIGS. 13 and 14 show perspective views of a saddle member of the dynamic screw assembly.

FIG. 15 shows a perspective view of the screw assembly in a partially assembled state.

FIG. 16 shows a perspective view of the assembly with the inner saddle member deviated to one side within an assembly housing.

FIG. 17 shows the assembly with the saddle member in a midline (“neutral”) position within outer housing.

FIG. 18 shows a cross-sectional view of the assembly with the inner saddle member positioned within the outer housing.

FIG. 19 shows an embodiment of a bone screw assembly.

FIG. 20 shows the bone screw assembly of FIG. 19 in an exploded state.

FIG. 21 shows a cross-sectional view of the assembly of FIG. 19.

FIGS. 22 and 23 schematically show alternative arrangements of magnets within orthopedic implants.

### DETAILED DESCRIPTION

Disclosed are devices and methods for the use of magnets in orthopedic prosthesis. While these device principles are illustrated in use within spinal implants, it should be appreciated that they can be used with any orthopedic device.

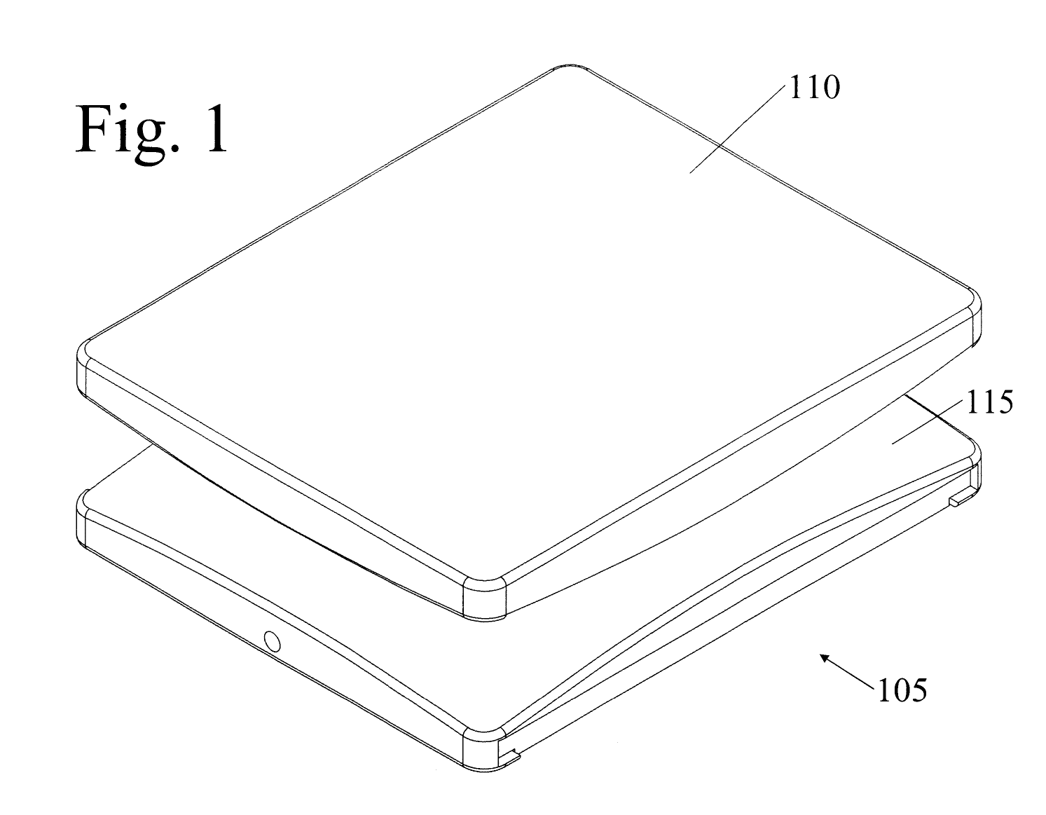

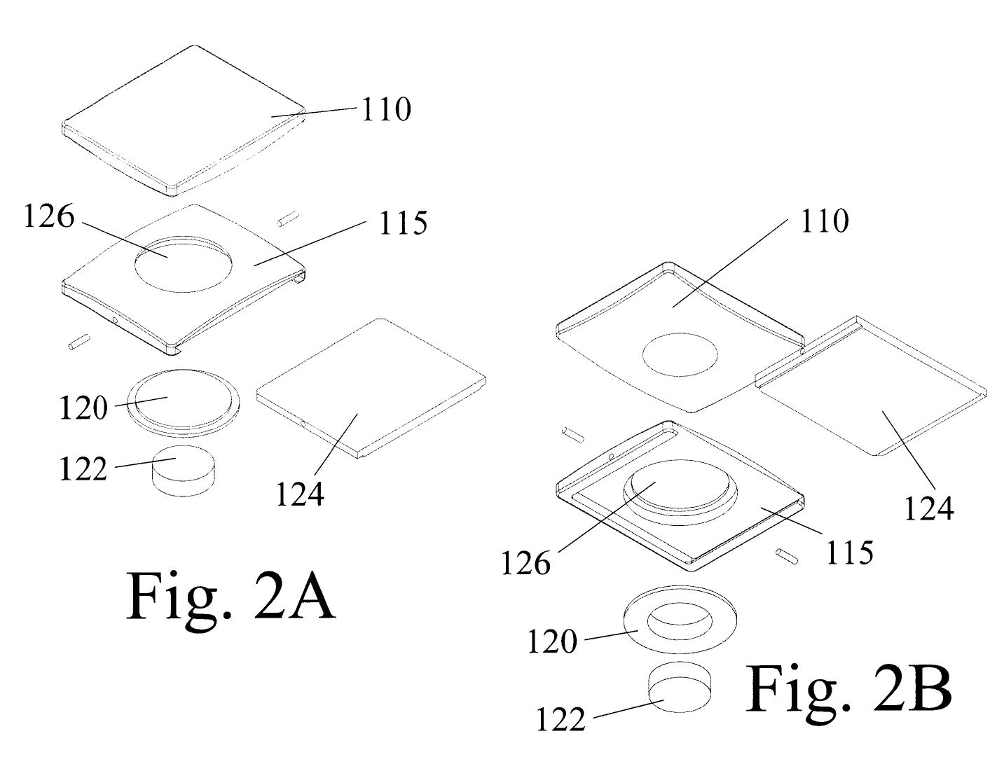

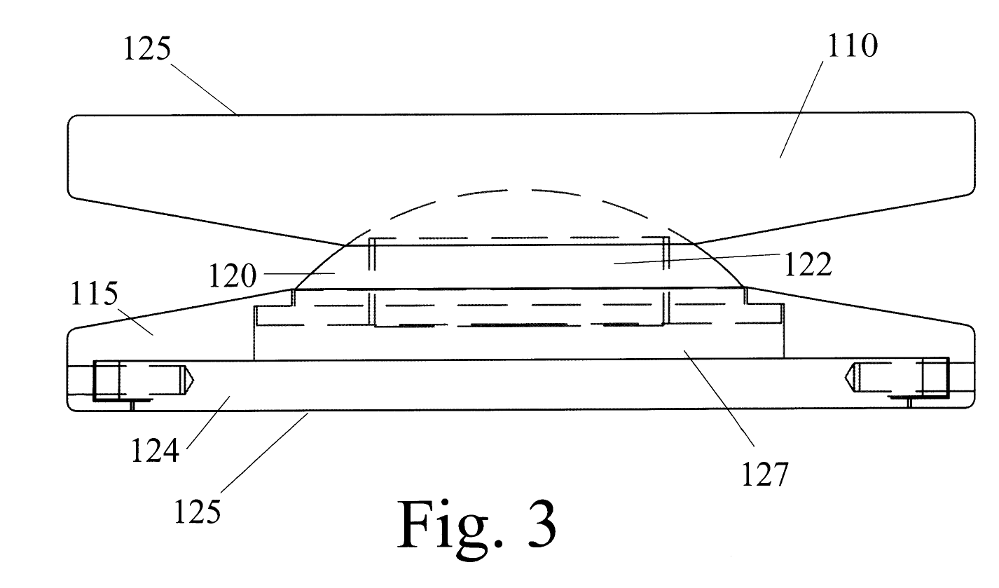

FIG. 1 shows a perspective view of a first embodiment of a prosthesis or implant 105 that is sized and shaped to be positioned within a disc space between a pair of vertebrae in a spine. FIGS. 2A and 2B show exploded views of the implant 105 and FIG. 3 shows a cross-sectional view of the implant of FIG. 1. The implant has two members that produce magnetic fields and are positioned with juxtaposed like polarity so that they repulse one another. The interaction of the magnetic fields is used to determine the position of the bearing surface in the vertical plane and thereby impart a shock absorption-like feature to the implant.

The implant 105 includes an upper component 110 and a lower component 115. A bearing component 120 is interposed between the upper and lower components and interacts with a complimentary spherical cut-out on component 110. A first magnet 122 is mounted within a seat in bearing component 120 and a second magnet 124 is mounted in the lower component 115. As shown in the cross-sectional views of FIG. 3, the magnet 124 is sized and shaped to fit within a complimentary-shaped seat within the lower component 115. It should be appreciated that the terms “upper” and “lower” are for reference purposes and use of such terms should not be limiting with respect to placement orientation.

The upper and lower components 110 and 115 each have an abutment surface 125 that is adapted to abut against a vertebra when the implant 105 is positioned in a disc space. The abutment surfaces 125 of the upper and lower components are preferably configured to promote interaction with the adjacent bone and affix the implant to the bone.

With reference to FIG. 3, a portion of the bearing component 115 is sized and positioned to move within a cavity 127 in the lower member 124. The bearing component 115 is movably mounted within the cavity 127 such that it can move in an up-and-down direction with respect to FIG. 3. The magnets interact with the bearing component 115 in a manner that influences movement of the bearing component within the cavity 127. For example, the magnetic fields can dampen movement of the bearing component so as to provide a shock-absorbing feature to movement of the bearing component within the cavity.

In use, an intervertebral disc is removed from the disc space between first and second (or upper and lower) vertebrae. After the inter-vertebral disc is removed, the implant 105 is placed into the evacuated disc space. The abutment surface 125 of the component 110 of the implant 105 abuts the lower surface of the upper vertebra while the abutment surface 125 of the lower component 115 abuts the upper surface of the lower vertebra. As mentioned, the abutment surface of each upper and lower component is preferably configured to promote interaction with the adjacent bone and affix the implant to it. For that purpose, the abutment surfaces may be textured, corrugated or serrated. They may be also coated with substances that promote osteo-integration such as titanium wire mesh, plasma-sprayed titanium, tantalum, and porous CoCr. The surfaces may be further coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, helical rosette carbon nanotubes or other carbon nanotube-based coating may be applied to the surfaces to promote implant-bone interaction.

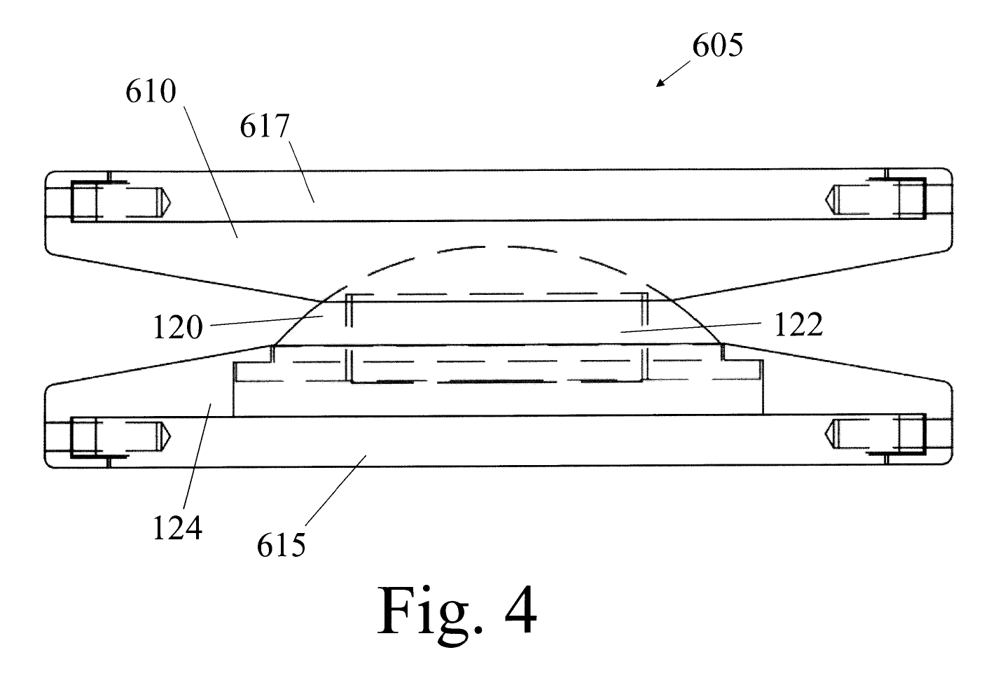

FIG. 4 shows another embodiment of an implant 605 that includes upper and lower components 610 and 615. The implant 605 is substantially similar to the previously-described implant 105. However, this embodiment includes a third magnet 617 within the upper component 610. The magnets are arranged such that like poles are aligned in the magnets 124 and 617. This causes the lower component and upper components to repel one another and lessen the load on the bearing surface. The bearing magnet 122 provides an additional magnetic force.

With reference to the embodiment of FIG. 4, the size of the magnetic fields produced by the magnets of the upper and lower components is selected to provide a predetermined interaction between the magnetic fields. For example, the magnetic forces may have a predetermined value or relationship to the amount of weight that is borne by the implant 105 when implanted in the disc space and that magnets of different strengths may be employed depending on the intended spinal region of implantation. In an embodiment, the implant is configured such that the repulsive magnetic forces between the upper and lower components are smaller than the weight borne by the implant 105 when the implant 105 is placed in the disc space. The upper and lower components are in contact when implanted and the force transmitted through the bearing component is necessarily less than the weight borne by the device. Alternatively, the magnetic force may be equal to or greater than the weight borne by the implant 105. The repulse force of the magnetic fields will work to partially off load or completely separate the bearing surfaces.

The implant 105 can exist in a neutral state. When in the neutral state, the various magnetic forces are in balance such that the upper and lower components are in a predetermined position relative to one another. The implant is preferably configured so that the neutral position provides an adequate distance between the upper and lower components and contact between the upper and lower components does not interfere with movement of the bearing component.

Movement of the implant away from the neutral position produces an imbalance in the sum of the magnetic forces. The implant resists movement away from the neutral position and returns the attached vertebral bones to the neutral position after the forces acting upon it have dissipated. In an alternative embodiment, the implant has an internal latch that prevents separation of the two members even when the weight borne is less than the repulsive force of the magnetic fields.

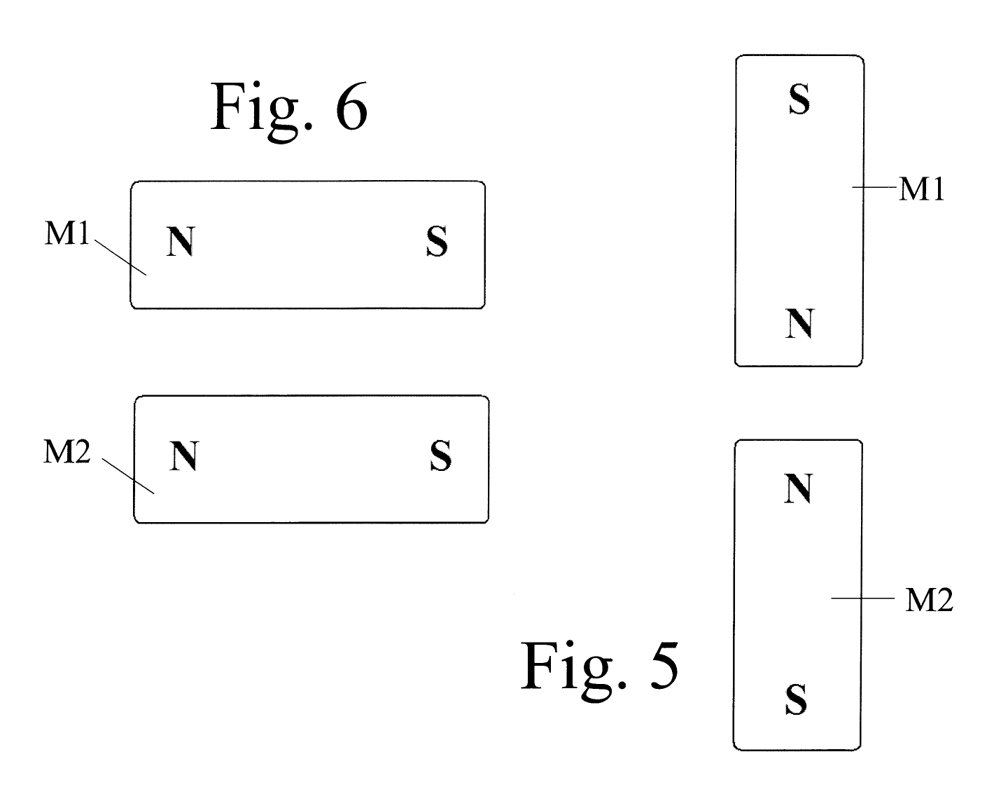

FIGS. 5 and 6 schematically illustrate the principles used in the preceding embodiments. FIG. 5 shows a first arrangement wherein like poles (north and north) are positioned adjacent to one another in an end-to-end configuration. The magnets will repel one another and the interaction of the fields will determine the relative position of the magnets in the vertical plane. The magnets are used to create a shock absorption-like feature in the prosthesis. This is similar to the design features employed in the first embodiment. FIG. 6 schematically shows a second arrangement wherein the magnets will resist movement away from the neutral position and returns the attached implant to the neutral position after the forces acting upon it have dissipated. This principle is illustrated in the embodiment of FIG. 4.

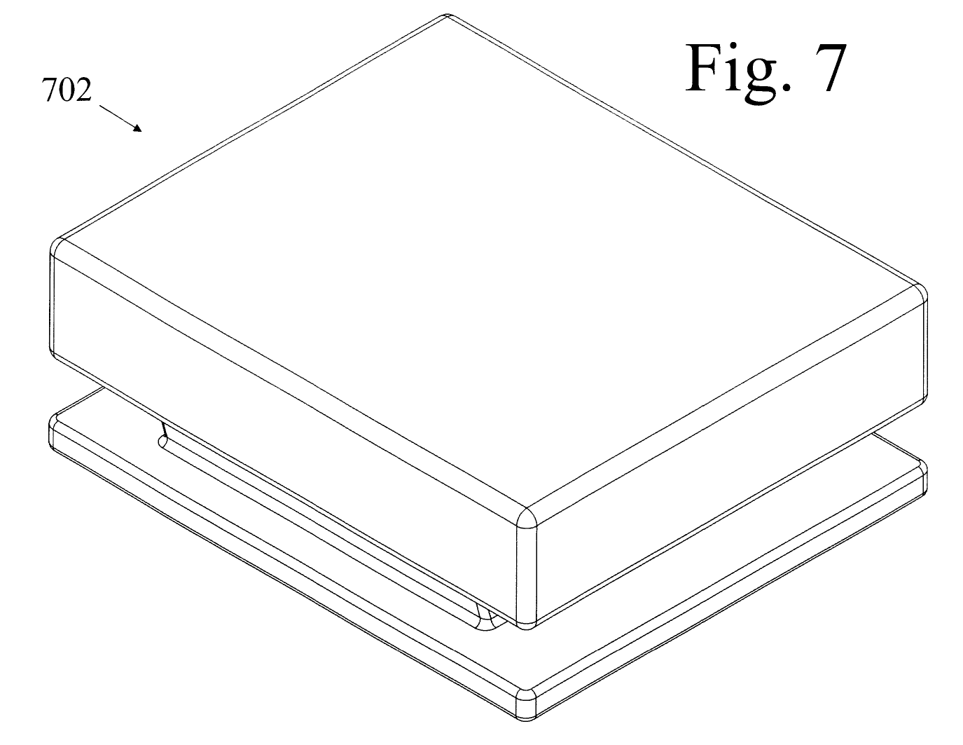

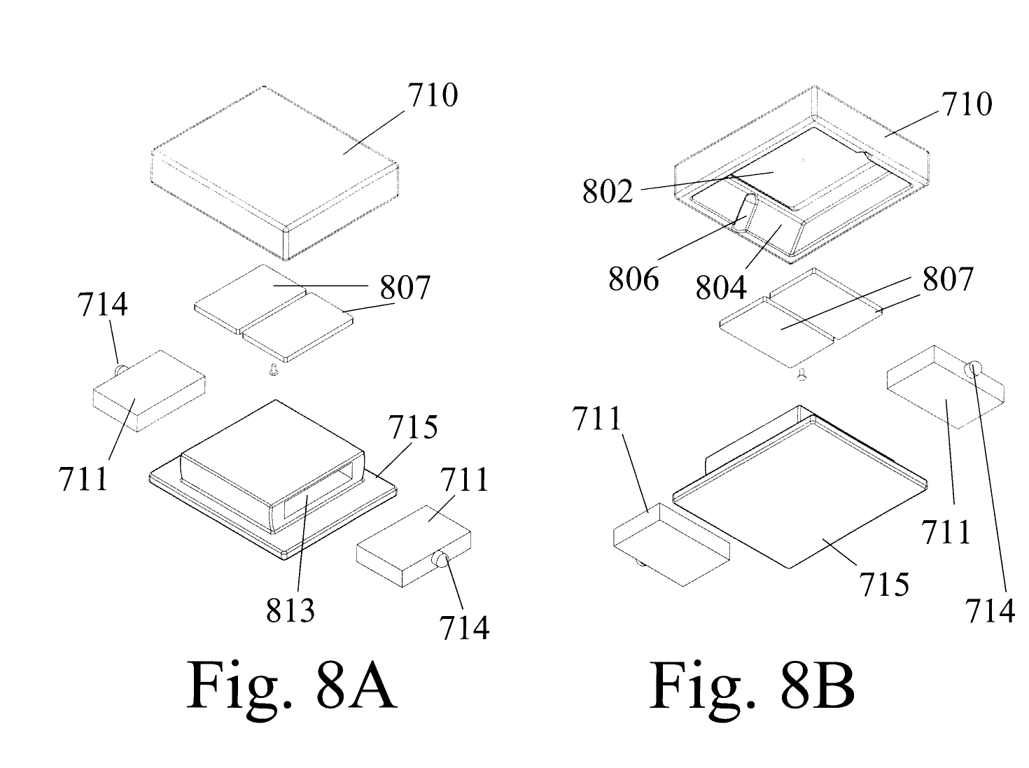

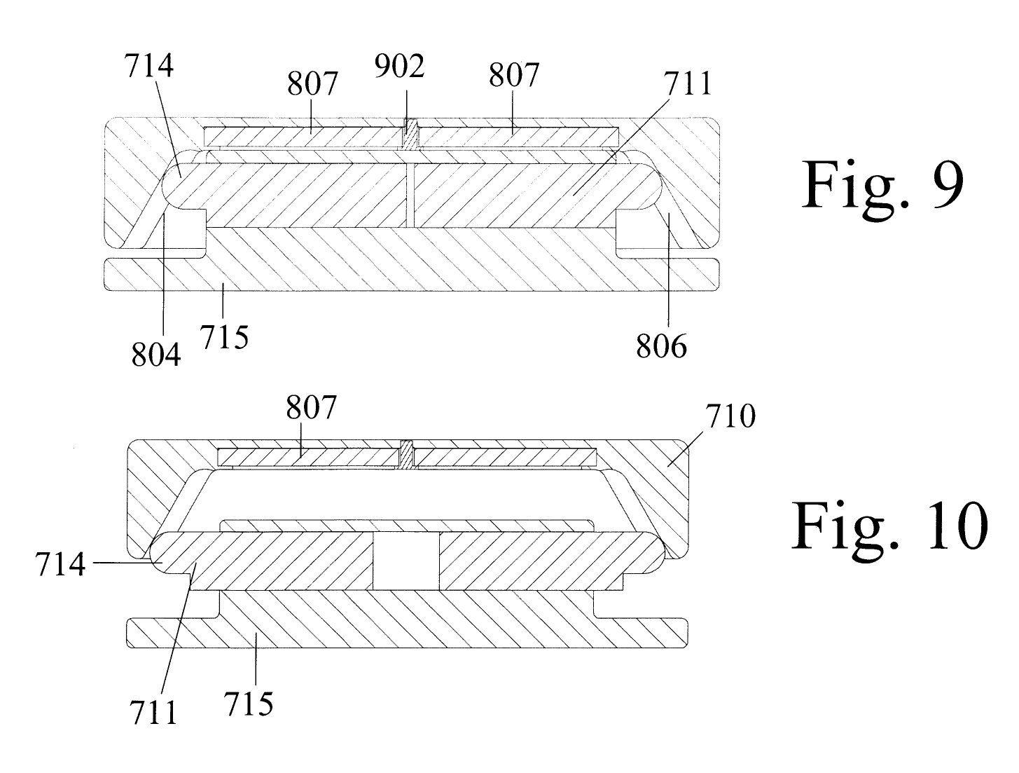

FIG. 7 shows another embodiment of an implant 702. FIGS. 8A and 8B shows exploded views of the implant 702 and FIG. 9 shows a cross-sectional view of the implant 702. The implant 702 includes an upper member 710 and a lower member 715. The upper member 710 has an internal cavity 802 (FIG. 8B) with sloped walls 804 having a channel 806. A pair of magnets 807 are adapted to be mounted within the upper component. A pair of magnets 711 with bearing members 714 are mounted within a cavity 813 in the lower member 715. The magnets partially or completely occupy the inner aspect of member 711. Members 711 are slidably positioned in cavity 813 and repulse outwardly away from one another, as described below.

With reference to the cross-sectional view of FIG. 9, the magnets 807 are mounted within the upper component 110 and secured therein, such as with a rivet 902. The lower component 115 is movably positioned below the upper component 110 with the magnets 711 positioned between the lower component 115 and the upper component 110. The bearing members 714 are positioned to abut the sloped walls 804 of the cavity 802. The magnets 711 are positioned with like poles adjacent one another such that the magnets 711 repulse one another outward toward the sloped walls 804. This forces the bearing components 714 to be forced against the sloped walls 804. Because the walls 804 are sloped, the bearing components 714 force the lower component 115 downwardly away form the upper component 110. The magnets 807 in the upper component 110 also repel the magnets 711 to provide further downward force to the lower component 115. FIG. 10 shows the lower component 115 in a full downward position relative to the upper component 110. The implant possess a shock absorption-like feature and, because of the parallel magnet configuration of magnets 87 and 711 (similar to that of FIG. 5), the device resists movement away from a neutral position and return the attached vertebral bones to that neutral position after the forces acting upon it have dissipated.

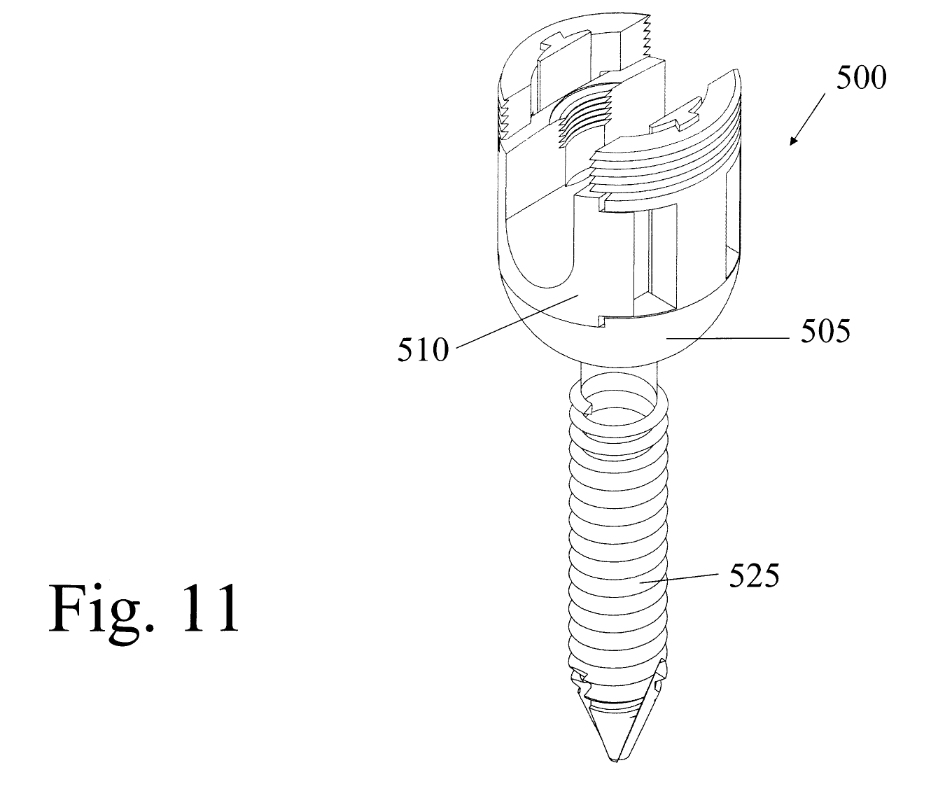

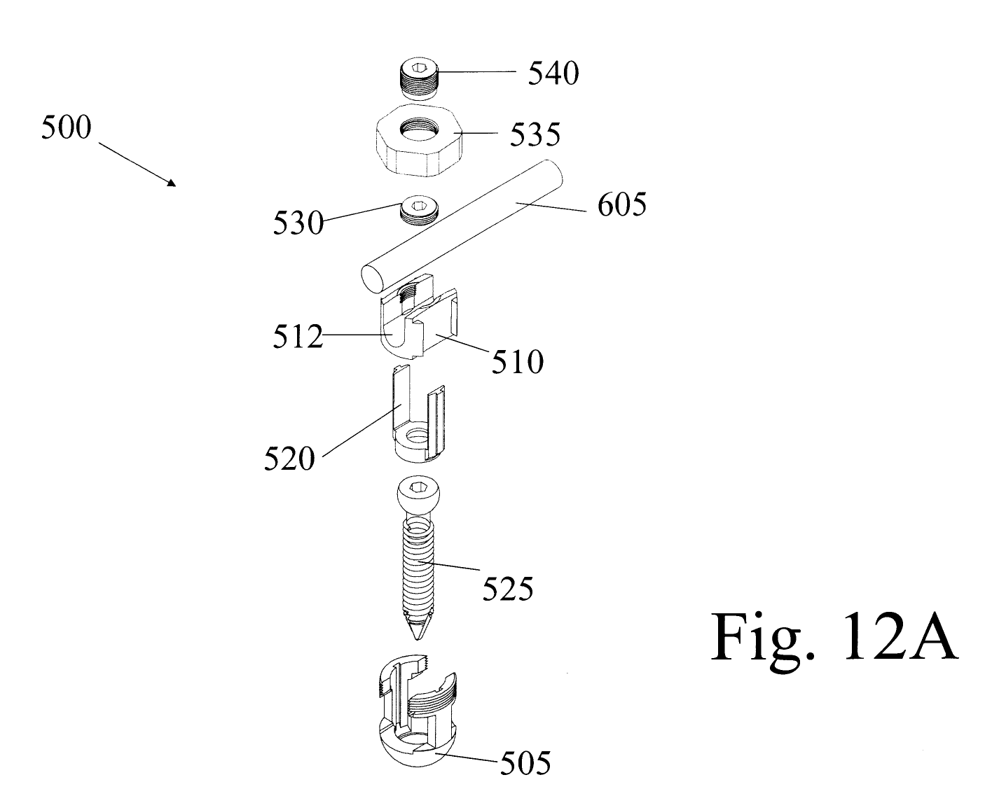

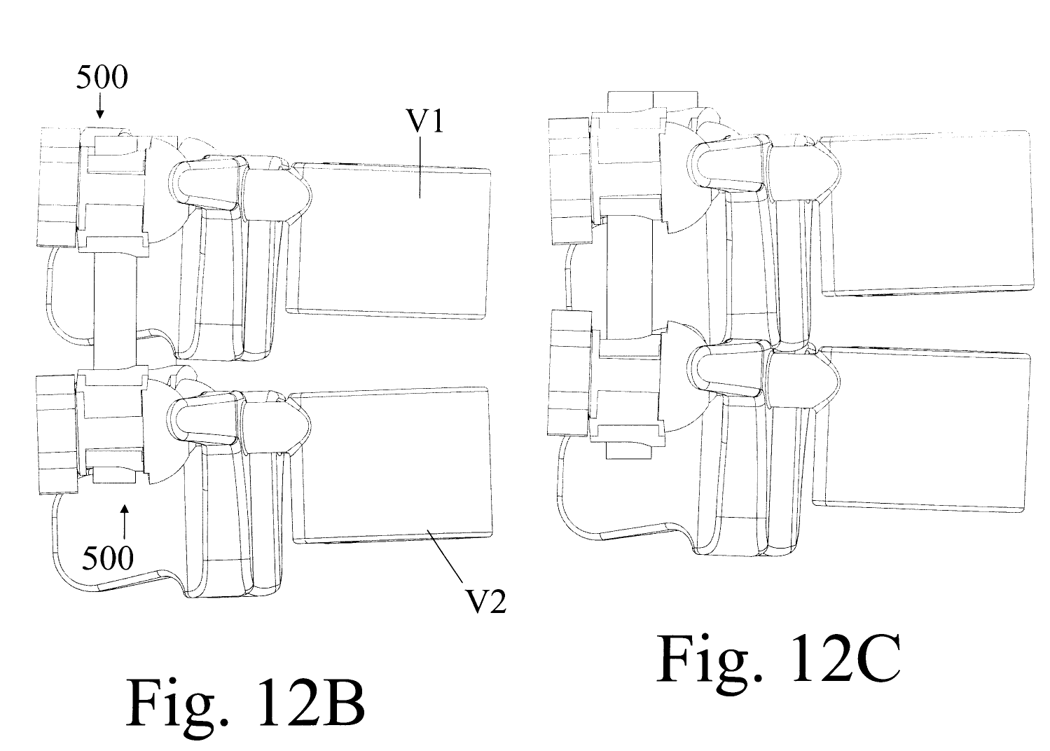

In another embodiment, the function of the facet joints of the first and second vertebrae of the spine may be modified or replaced using a dynamic screw assembly. FIG. 11 shows an assembled view of a bone screw assembly 500 that permits movement of a screw, rod, and/or housing relative to one another prior to complete locking of the device. FIG. 12A shows an exploded view of the assembly of FIG. 11. FIGS. 12B and 12C show dynamic screw assemblies attached to vertebral bodies V1 and V2 and linked via a rod 605. FIG. 12B show the vertebral bodies in flexion while FIG. 12C shows the vertebral bodies in extension.

The assembly of FIGS. 11 and 12A includes a housing that is formed of several components that can move or articulate relative to one another. The rod can be immobilized relative to a first component while the screw can be immobilized relative to a second component of the housing. Because the first and second components are movable relative to one another, the rod and screw can move relative to one another while still being coupled to one another.

With reference to FIGS. 11 and 12A, the assembly 500 includes a housing comprised of an outer housing 505 and an inner saddle member 510 having a slot 512 for receiving a rod 605 (FIG. 12). A locking member 520 (FIG. 12) fits within the outer housing 505 above a bone screw 525. The bone screw 525 sits within a seat in the bottom of the outer housing 505 such that a shank of the screw 525 extends outwardly from the outer housing 505. An inner locking nut 530 interfaces with the saddle member 510 for providing a downward load on the rod 615 for securing the rod relative to the saddle member 510, as described below. An outer locking nut 535 interfaces with the outer housing 505 for locking the assembly together, as described below. A central locking nut 540 engages a central, threaded bore within the outer locking nut 535. The locking nuts 530, 535, and 540 can provide various combinations of immobilization of the rod 615, screw 625, and housing relative to one another.

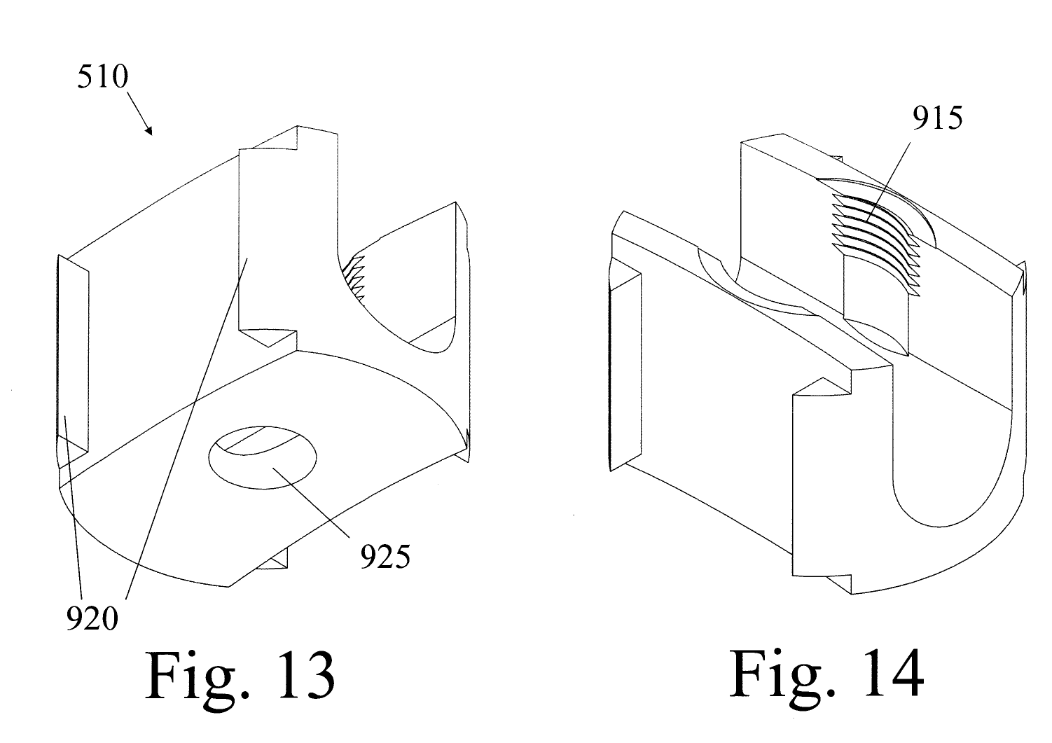

FIGS. 13 and 14 show perspective views of the saddle member 510. The saddle member 510 has a pair of opposed extensions 905 that form a rod channel 910 therebetween wherein the channel 910 is adapted to receive the rod 615. A threaded engagement region 915 on the inner surface of the extensions 905 is adapted to interface with the inner locking nut 530 (FIG. 12). The outer aspect of each extension 905 includes a pair of protrusions 920 that function to limit the amount of movement of the saddle 510 relative to the outer housing 505 of the assembled device, as described in detail below. A borehole 925 extends through a base of the saddle member 510.

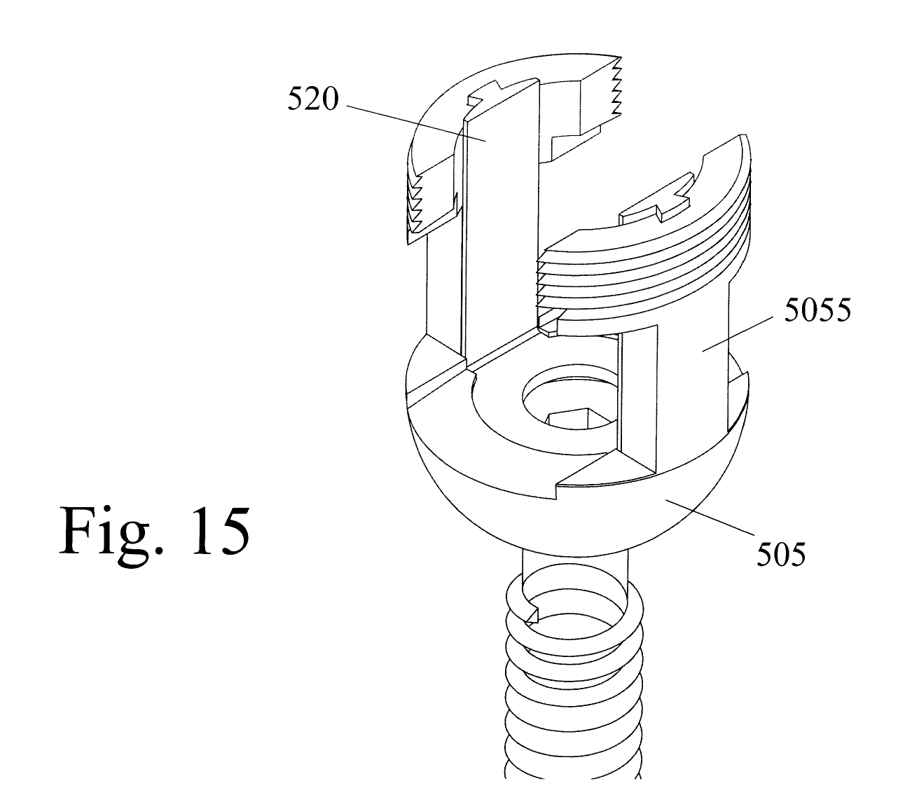

FIG. 15 shows a perspective view of the assembly 500 in a partially assembled state with the screw 525 and the locking member 520 engaged with the outer housing 505. The head of the screw 525 is positioned within a seat in the outer housing 505 such that the shank of the screw 525 extends through a bore in the outer housing 505. The screw head is free to move within the seat. That is, the head can rotate within the seat in a ball and socket manner. The locking member 520 is positioned within the outer housing such that upper edges of the locking member 520 can be pressed downwardly so that the locking member 520 exerts a locking force on the screw head to immobilize the screw 525 relative to the outer housing 505. The outer locking nut 535 can be used to press the upper edges of the locking member 520 downward.

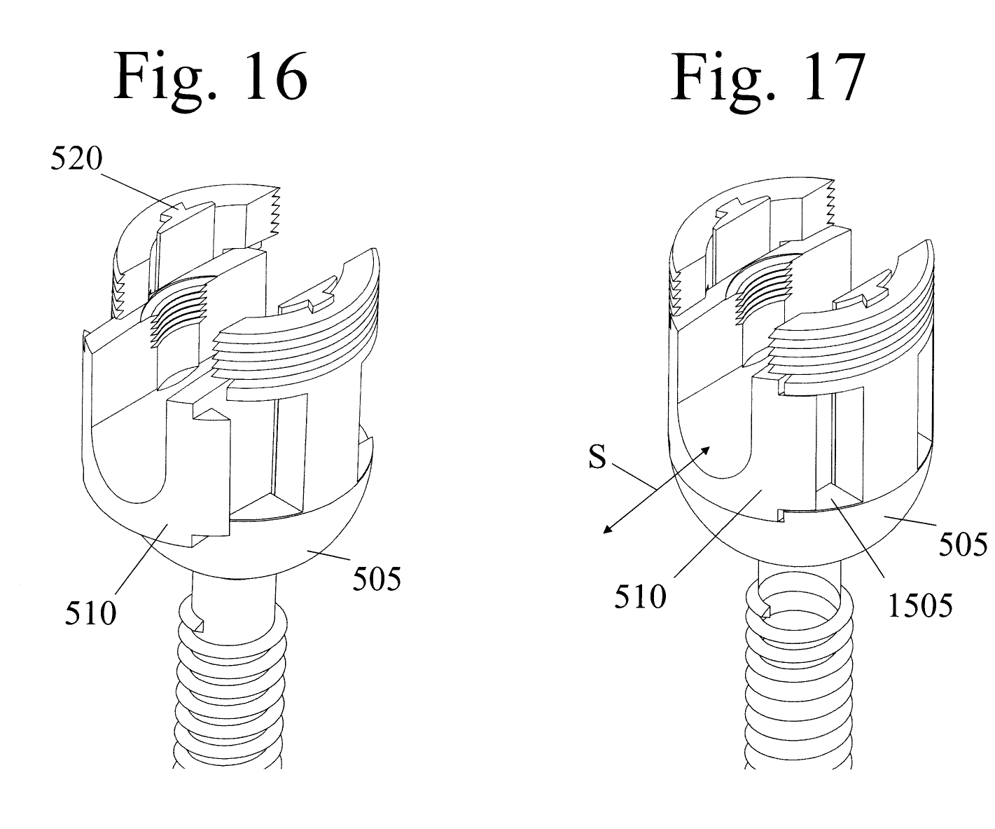

FIG. 16 shows a perspective view of the assembly with the inner saddle member 510 deviated to one side within housing 505. FIG. 17 shows the assembly with the saddle member 510 in a midline (“neutral”) position within outer housing 505. The saddle member 510 slides into the space between upward extensions on the outer housing 505 and the locking member 520. With reference to FIG. 17, a space 1505 is located between the inner saddle member 510 and the housing 505. The spaces 1505 permit the saddle member 510 to have some play or movement relative to the outer housing 505 when the saddle member 510 is positioned in the outer housing 505.

It should be appreciated that the size and shape of the spaces 1505 can be varied. Moreover, the saddle member 510 can be sized and shaped relative to the outer housing 505 such that other spaces are formed. At least one purpose of the spaces is to permit relative movement between the saddle member 510 and the outer housing 505 and this can be accomplished in various manners. Thus, the screw can be moved from a first orientation (such as the neutral position) to a second orientation while the rod is immobilized relative to the inner member 510.

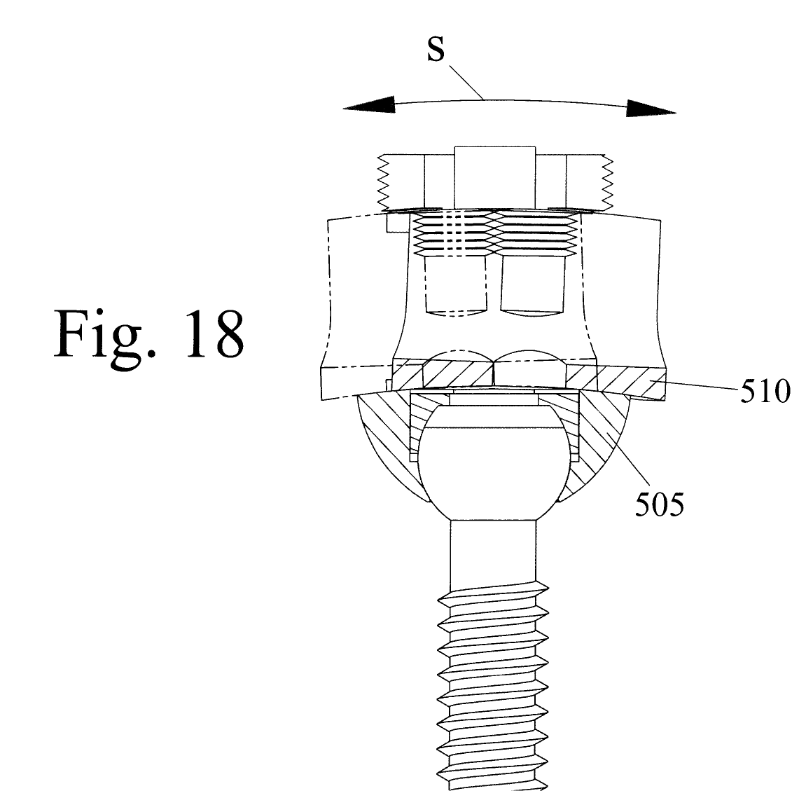

The inner saddle member 510 can slidably move within the outer housing 505 along a direction aligned with axis S wherein the amount movement is limited by the interplay between the inner saddle member and outer housing. This type of movement is represented in FIG. 18, which shows a cross-sectional view of the assembly with the inner saddle member 510 positioned within the outer housing 505. The inner saddle member 510 is represented in solid lines at a first position and in phantom lines at a second position after sliding from right to left in FIG. 18. The bottom surface of the inner saddle member slides along the upper surface of the outer housing 505. As mentioned, the surfaces can be contoured such that the inner saddle member slides along an axis S that has a predetermined radius of curvature. This can be advantageous during flexion and extension of the attached spinal segments, as the radius of curvature of the axis S can be selected to provide motion along the physiologic axis of rotation of the spinal segments.

In one embodiment, protrusions 920 of saddle member 510 as well as central post 5055 outer housing 505 can be fitted with (or made out of) members capable of producing a magnetic field. The magnetic members are positioned with like polarity facing one another so that the components repel each other. While the device permits movement of the inner saddle member 510 relative to the housing 505, the repulsive magnetic fields of the saddle member and the housing resist any movement away from the neutral position and return the assembly to neutral after the force producing the movement has dissipated. The interaction of the magnetic fields influences the extent of rotation and translation of members of the assembly.

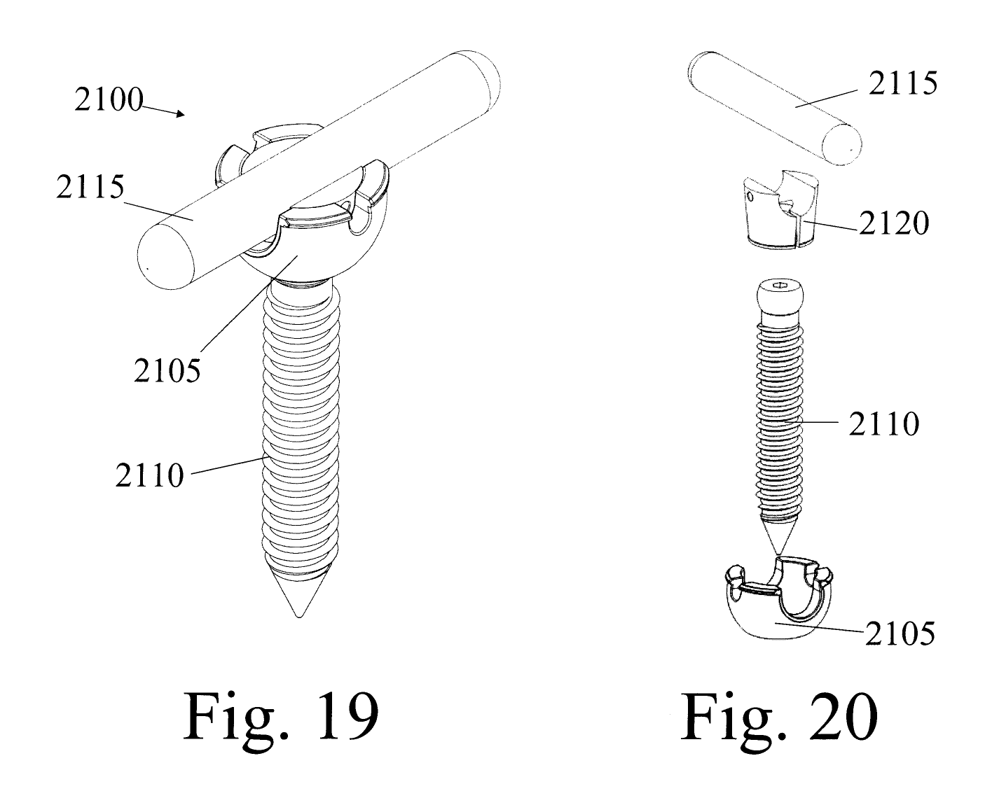

FIG. 19 shows an embodiment of a bone screw assembly. FIG. 20 shows the bone screw assembly of FIG. 19 in an exploded state. The bone screw assembly 2100 includes a housing 2105, a bone screw 2110 that fits through a bore in the housing 2105, and a rod 2115. The rod 2115 lockingly engages a pair of locking members 2120.

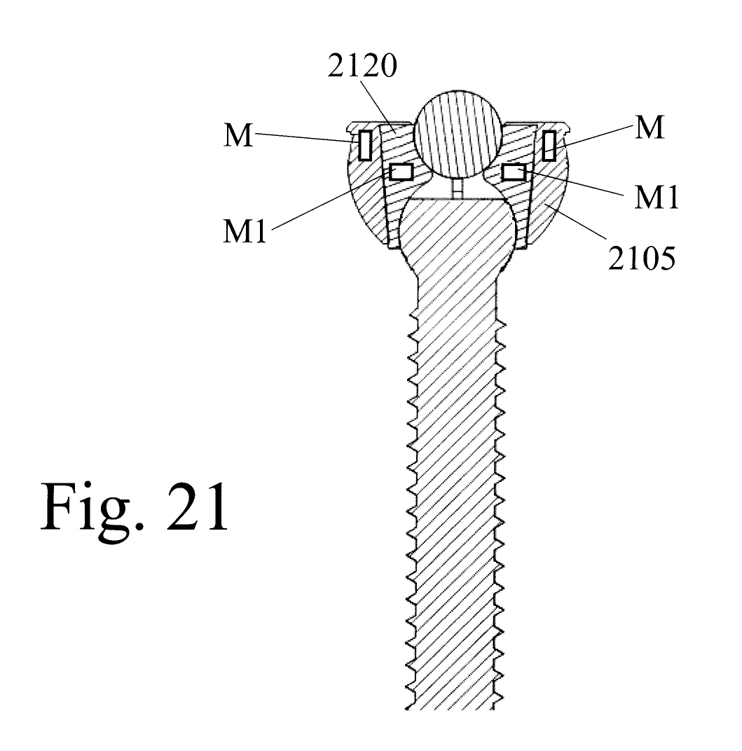

FIG. 21 shows a cross-sectional view of the assembly of FIG. 19. The locking members 2120 can lock to the housing 2105 and the rod 2115 using a Morse taper configuration. When the locking members 2120 are pressed downward into the housing 2105 by the rod 2115, the two locking members 2120 are forced inward toward the rod 2115 to immobilize the rod 2115 therebetween. With the assembly in the locked configuration, the outer surfaces of the locking members 2120 tightly fit within the inner surface of the housing 2105. The individual segments of the locking members 2120 are forced inward and immobilize the rod 2115 and the rotational members 3125 relative to one another. In this way, the assembly serves to lock the rod 2115 relative to the bone screw 2110.

Although a Morse taper locking mechanism provides a powerful immobilization, it may be loosened with only a modest backout of the locking members 2120 relative to the housing 2105. This may be prevented by the addition of a magnetic locking mechanism. One or more magnet components M and M1 can be positioned within the locking member(s) 2120 and/or housing 2105, as shown in FIG. 21. In one embodiment, one or more magnets Ml are positioned within the locking members 2120 while one or more magnets M are positioned within the housing 2105. Magnets M and M1 are positioned with like polarity facing one another such that the magnets repel one another. With the screw assembly locked, magnet M is positioned above magnet M1 in the vertical plane. Loosening of the device requires that magnet M1 move towards magnet M and this movement will be opposed by the repulsive force of the magnetic fields.

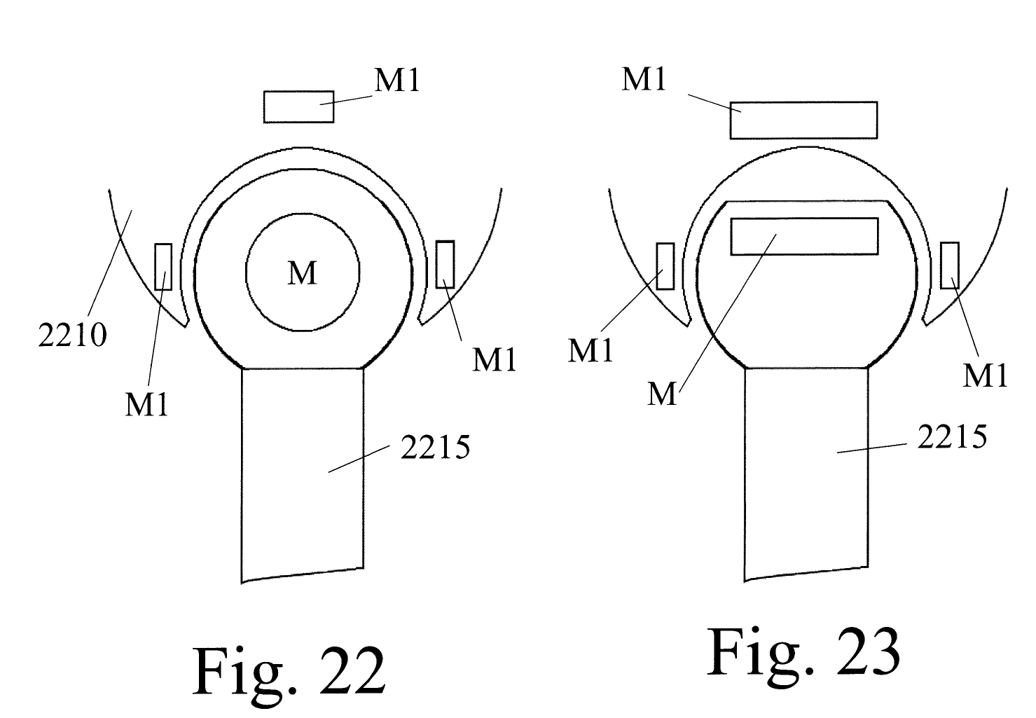

FIGS. 22 and 23 schematically show the use of magnets in an orthopedic implant that has a ball and socket configuration. The implant 2205 of FIG. 22 has a first component 2210 attached to a first bone structure and a second component 2215 attached to a second bone structure. The first and second components interface with one another in a ball and socket manner. The component 2215 has one or more magnets M mounted therein or is alternately manufactured or partially manufactured of a magnetic material. The component 2210 similarly is configured with magnets M1. The magnets M1 are situated around the ball and socket structure to provide predetermined magnetic interaction between the two components. In this configuration, the interaction of the magnetic fields will reduce the contact between the two components across the ball and socket joint. Further, placement of the magnets in the configuration shown in FIG. 23 will allow the implant to resist movement away from a neutral position and returns the attached bones to that neutral position after the forces acting upon it have dissipated.

Finally, the fields generated by the magnetic members of the implant may have pain reducing effects on neighboring tissues. These fields will bath neighboring tissues and may provide an additional benefit and advantage over orthopedic implants that do not contain magnets. Since magnetic fields of varying strength are believed to have greater tissue effect than fields with constant strength, the devices may be configured so that the neighboring tissues are exposed to a variable magnetic filed. In an embodiment, a member of the prosthesis that is mobile relative to the magnetic field source can be fitted with magnetically shielding material and positioned between the field source and the target tissue. With normal prosthesis movement, the shielding member will move between the magnetic member and the surrounding tissues and the tissues will experience a variation in the magnetic field.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed:

1. An orthopedic device adapted to be implanted between a first bone and a second bone of a skeletal structure, comprising:
a first member having an abutment surface adapted to contact a surface of the first bone, wherein the first member emits a first magnetic field of a first polarity;
a second member having an abutment surface adapted to contact a surface of the second bone, wherein the second member emits a second magnetic field of the same polarity as the first polarity; and
at least one bearing member between the first and second members that permits relative movement between the first and second members and that bears a load between the first and second members, wherein the load on the bearing surface is reduced as a result of an interaction of the magnetic fields.

2. A device as in claim 1, wherein the first and second bones are first and second vertebrae.

3. A device as in claim 1, wherein at least one of the first and second members is partially manufactured of a magnetic material.

4. A device as in claim 1, wherein at least one of the first and second members is entirely manufactured of a magnetic material.

5. A device as in claim 1, wherein a magnet is removably mounted in at least one of the first and second members.

6. An orthopedic device adapted to be implanted between a first bone and a second bone of a skeletal structure, comprising:
a first abutment member having an abutment surface adapted to contact a surface of the first bone;
a first magnetic member at least partially contained within the first abutment member, wherein the first magnetic member emits a first magnetic field of a first polarity;
a second abutment member having an abutment surface adapted to contact a surface of the second bone; and
a second magnetic member at least partially contained within the second abutment member, wherein the second magnetic member emits a second magnetic field of the same polarity as the first polarity;
wherein the first and second abutment members have a spatial relationship that is at least partially determined by an interaction of the first and second magnetic fields.

7. A device as in claim 6, wherein the first and second abutment members can translate relative to one another and wherein the extent of translation is at least partially determined by an interaction of the first and second magnetic fields.

8. A device as in claim 6, wherein the first and second abutment members can rotate relative to one another and wherein the extent of rotation is at least partially determined by an interaction of the first and second magnetic fields.

9. An orthopedic device adapted to be implanted between a first bone and a second bone of a skeletal structure, comprising:
a first abutment member having an abutment surface adapted to contact a surface of the first bone;
a first magnetic member at least partially contained within the first abutment member, wherein the first magnetic member emits a first magnetic field;
a second abutment member having an abutment surface adapted to contact a surface of the second bone; and
a second magnetic member at least partially contained within the second abutment member, wherein the second magnetic member emits a second magnetic field;
wherein the first and second abutment members have a default spatial relationship and wherein movement of the first and second members away from the default spatial relationship is opposed by interaction of the first and second magnetic fields.

10. A device as in claim 9, wherein the first and second magnetic fields are of the same polarity.

11. A device as in claim 9, wherein the first and second magnetic members attract one another.

12. A device as in claim 9, wherein the first and second magnetic members repel one another.

13. An orthopedic device adapted to be implanted between a first bone and a second bone of a skeletal structure, comprising:
a first abutment member having an abutment surface adapted to contact a surface of the first bone;
a first magnetic member at least partially contained within the first abutment member, wherein the first magnetic member emits a first magnetic field; and
a second abutment member having an abutment surface adapted to contact a surface of the second bone;
a second magnetic member at least partially contained within the second abutment member, wherein the second magnetic member emits a second magnetic field;
wherein the first and second members can move relative to one another and wherein relative movement between the first and second members is at least partially hindered by interaction of the magnetic fields.

14. A device as in claim 13, wherein the first and second magnetic members attract one another.

15. A device as in claim 13, wherein the first and second magnetic members repel one another.

16. An orthopedic device adapted to be implanted in a patient, comprising:
a first member having an abutment surface adapted to attach to a surface of a bone so as to aid in segmental stabilization of the patient's skeletal system; and
a first magnetic member at least partially contained within the first abutment member, wherein the first magnetic member emits a first magnetic field such that the magnetic field reaches a tissue of the patient.
